(19) United States
(12) Patent Application Publication
(10) Pub. No.: US 2004/0199185 A1
(43) Pub. Date: Oct. 7, 2004

(54) Suturing method and apparatus

(75) Inventor: Barry Davignon, Terre Haute, IN (US)

Correspondence Address: David H. Badger, Esq., BRINKS HOFER GILSON & LIONE, One Indiana Square, Suite 1600, Indianapolis, IN, 46204, US

(73) Assignees: Rose-Hulman Institute of Technology; Rose-Hulman Ventures

(21) Appl. No.: 10/772,500

(22) Filed: Feb. 5, 2004

Related U.S. Application Data
(60) Provisional application No. 60/449,044, filed on Feb. 21, 2003.

(51) Int. Cl. A61B 17/04

(52) U.S. Cl. 606/148; 606/139; 606/222

(57) ABSTRACT

Sutures can be reliably applied to the tissue of sensitive organs in a predetermined and limited depth with minimal surgical skills through the use of a curved suture needle and a suture control apparatus. The suture control apparatus, which can be adjustable, comprises a pair of needle guides that are separated by a space, and a locator surface for engagement with the suture-receiving tissue in the space between the needle guides, with the pair of needle guides and locator surface being preferably carried by a handle. Preferably, the pair of needle guides are curved to provide a radius of curvature substantially equal to the radius of curvature of the curved suture needle. The radius of the curvature of the suture needle and curved needle guides, the distance of the space between the pair of curved needle guides and the location and shape of the locator surface, when pressed against tissue to receive a suture, provide a predetermined and limited depth of penetration of a suture needle and suture, when the curved suture needle is threaded through the pair of curved needle guides and the tissue in the space therebetween.

### RELATED APPLICATIONS

The present patent document claims the benefit of the filing date under 35 U.S.C. §119(e) of Provisional U.S. Patent Application Serial No. 60/449,044, filed Feb. 21, 2003, which is hereby incorporated by reference.

### FIELD OF THE INVENTION

This invention relates to an apparatus and method for the suturing operations and, more particularly, to an apparatus and method by which the depth of the penetration of the sutures may be limited in suturing sensitive tissues, such as the tissues of the eye.

### BACKGROUND OF THE INVENTION

In many operations it is necessary to suture the tissues of different parts of the body together, and in many cases, the body parts are sensitive and may be severely injured in the application of the attaching sutures if the suture needle penetrates too deeply into the tissue of the body part. For example, in the correction of strabismus (such as cross eye or walleye), the eye muscle is detached from the eyeball, which is being held in a misaligned position, correctly located and reattached to the eyeball through the application of sutures. The tissue of the eyeball, like many body parts, is sensitive, and unfortunately there is a danger that during the application of sutures reattaching the muscles to the eyeball, the suturing needle can penetrate into the eyeball tissue deeply enough to injure the eyeball and the sight of the patient.

Accordingly, there is a need for a suturing apparatus and method that can be used to reliably control the maximum depth penetration of sutures during a suturing operation and remove the danger of injury to the patient by even the most skilled and careful surgeons.

### SUMMARY OF THE INVENTION

The invention permits sutures to be reliably applied to the tissue of sensitive organs in a predetermined and limited depth with minimal surgical skills.

In the invention, a suture control apparatus is provided for use with a curved suture needle. The suture control apparatus comprises a pair of needle guides that are separated by a space, and a locator surface for engagement with the suture-receiving tissue in the space between the needle guides, with the pair of needle guides and locator surface being preferably carried by a handle. Preferably, the pair of needle guides are formed from short lengths of tubing providing a radius of curvature substantially equal to the radius of curvature of the curved suture needle. The radius of the curvature of the suture needle, the distance of the space between the pair of needle guides and the location and shape of the locator surface, when pressed against tissue to receive a suture, can control and provide a predetermined and limited depth of penetration of a suture needle and suture, when the curved suture needle is threaded through the pair of curved needle guides and the tissue in the space between them.

The invention thus provides a method of providing sutures having predetermined maximum depth of penetration in tissue by providing a curved suture needle having a radius of curvature and an attached suture material; providing a suture control apparatus for the curved suture needle, comprising a pair of needle guides, which are preferably curved, separated by a space with a locator surface at a pre-selected site in the space between the pair of curved needle guides, the curved needle guides providing the same radius of curvature as the radius of curvature of the curved suture needle; pressing the tissue to receive a suture against the locator surface in the space between the pair of curved needle guides and locating the pair of curved needle guides adjacent the portion of the tissue to receive the suture; and threading the suture needle and suture material through the pair of curved needle guides and the tissue therebetween to provide a suture having a predetermined depth of penetration of the tissue, which has been determined by the radius of curvature of the suture needle, the distance between the pair of curved needle guides and the location and shape of the locator surface of the suture control apparatus. This apparatus and method can be used in any surgical procedure that will allow the tissue or tissues to be seated on the locator surface with sufficient clearance for the needle to pass through the needle guides.

More particularly, the invention provides an apparatus and method for permitting sutures to connect the muscles which control the location and movement of the eyeball to the tissue of an eyeball, by providing a curved suture needle having a radius of curvature and a suture control apparatus for the curved suture needle, the suture control apparatus comprising a pair of curved needle guides providing the same radius of curvature as the curved suture needle and being separated by a space which includes a locator surface having a curved portion with the same radius as the eyeball at a pre-selected location between the pair of curved needle guides; and seating the locator surface of the suture control apparatus on the surface of the eyeball in the space between the pair of curved tubular needle guides to limit the depth of penetration of the suture needle and suture into the sensitive eyeball tissue when they are threaded through the curved needle guides.

Other features and advantages of the invention will be apparent to those skilled in the art from the accompanying drawings and the more detailed description of the invention that follows.

### BRIEF DESCRIPTION OF THE DRAWINGS

FIG. 1 includes perspective views of a suture control apparatus and suture needle of the invention;

FIGS. 2 through 6B are diagrams to illustrate the invention and its method of use, in which:

FIG. 2 is a diagram of an apparatus of the invention prior to its use;

FIG. 3 is a diagram showing a suture control apparatus of the invention seated on an eyeball;

FIG. 4 is a diagram showing a suture control apparatus of the invention and suture needle in the process of applying a suture to an eyeball;

FIG. 5 is a diagram showing a suture control apparatus and suture needle having completed the application of a suture through the tissue of an eyeball;

FIGS. 6A and 6B are expanded views of the curved needle guides of the suture control apparatus to illustrate slots in the needle guides permitting removal of the suture control apparatus following the application of a suture, FIG. 6B showing the disengagement of the suture control apparatus from a suture after its application;

FIGS. 7A-7C are diagrammatic drawings of other embodiments of suture control apparatus and suture needles comprising apparatus and methods of the invention; and

FIGS. 8A and 8B illustrate a further embodiment of the invention which is adjustable and permits variation in the controlled depth of penetration of a suture; FIG. 8A illustrating the suture control apparatus from its front; and FIG. 8B. illustrating the suture control apparatus from its back.

### DETAILED DESCRIPTION OF THE DRAWINGS AND THE PRESENTLY PREFERRED EMBODIMENTS

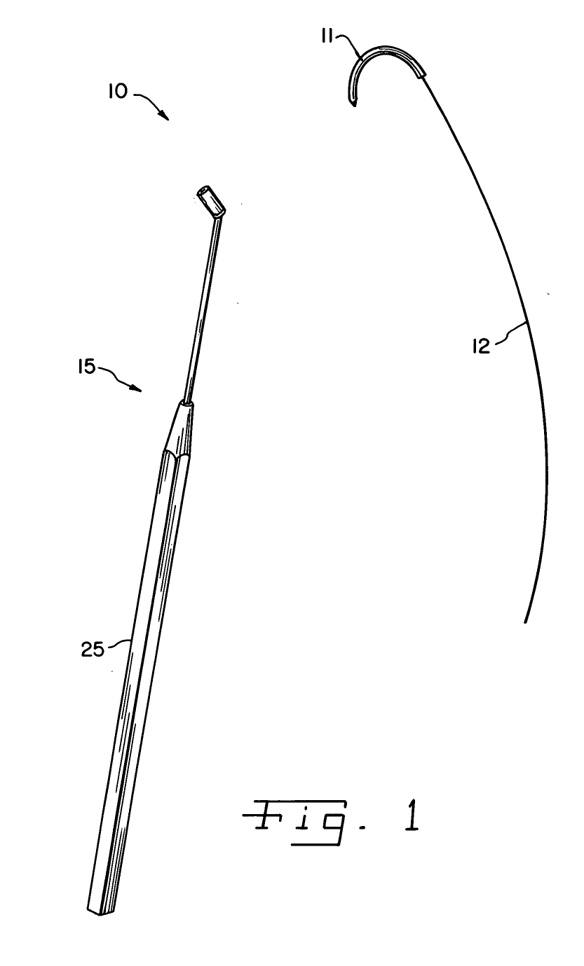

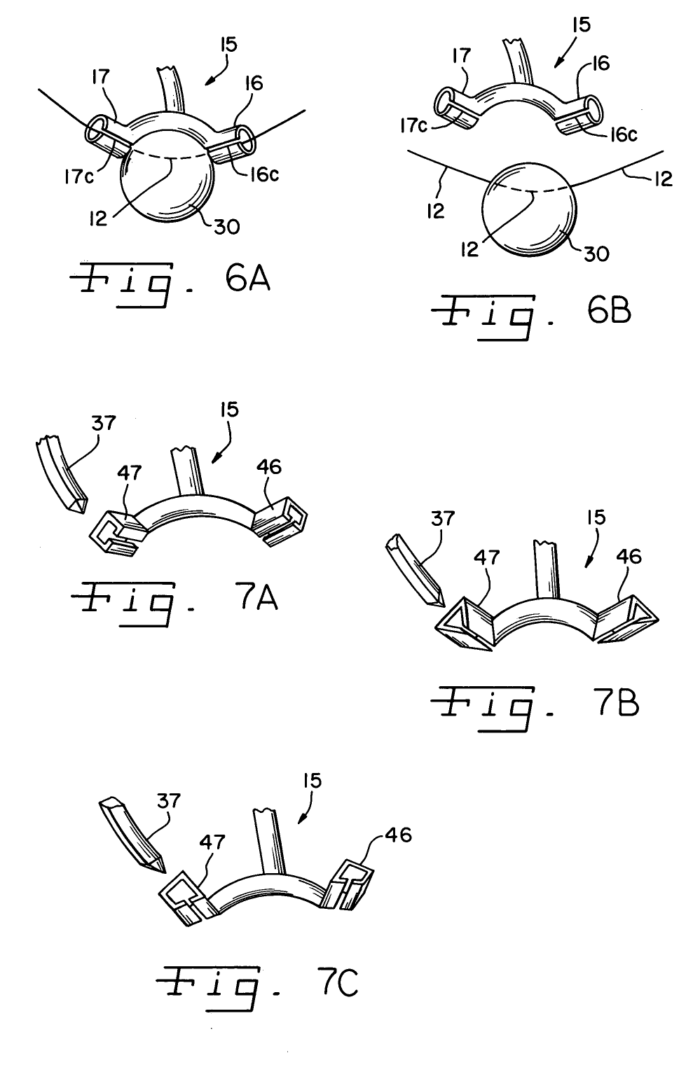

FIG. 1 illustrates one embodiment of an apparatus of the invention. As illustrated in FIG. 1, the apparatus 10 of the invention includes a curved suture needle 11 and a suture control apparatus 15. As illustrated in the FIG. 2 diagram, the suture control apparatus preferably includes a pair of curved tubular needle guides 16, 17 having outer openings 16a, 17a, and inner openings 16b, 17b and slots 16c, 17c (shown in FIGS. 6A and 6B). A locator surface 20 is provided in the space 18 between the inner openings 16b, 17b of the curved tubular needle guides 16 and 17. Preferably, the pair of curved tubular needle guides 16, 17 and locator surface 20 are attachable to and carried by a handle 25. Preferably, the radius of the curvature of the suture needle 11 and the radius of curvature of the curved needle guides 16, 17 are substantially equal. In addition, the inside diameters of the curved tubular needle guides 16, 17 are slightly larger than the outside diameter of the suture needle 11 so that suture needle 11 may pass freely through the curved tubular needle guide 16, 17 with little or no friction.

For best control of the depth of penetration of a suture, it is desirable, however, that the walls forming the needle passageways of the curved needle guides can engage the outside surface of the curved needle sufficiently to avoid excessive rotation and lateral movement of the curved suture needle within the curved needle guides. While the illustrated curved needle guides 16, 17 are shown as formed from short lengths of tubing, one or both of the curved needle guides may be formed from tubing which has a partially open portion, or open portions, that are intermediate the portions forming the inner and outer openings of the curved needle guides so long as the curved needle guides engage the outside surface of the curved suture needle sufficiently to avoid excessive rotation or lateral movement of the curved suture needle within the curved needle guides. For most curved suture needles the length of the curved needle guides between the outer and inner openings need only be a small fraction of an inch, e.g., about ⅛ inch to about ¼ inch. An excessive clearance between the outer surface of the suture needle 11 and the inside diameter of the curved tubular needle guides 16, 17 can also permit sutures which are deeper or shallower than intended or laterally displaced from their intended location because the suture needle 11 may rotate slightly within the curved needle guides or be displaced from concentricity from the curved needle guides 16, 17. In addition, while the slots 16c 17c can be located in any part of the periphery of the needle guides, it is preferred that they be located in the sides of the needle guides as shown in FIGS. 6A and 6B. A slot located in the bottom of the needle guide may affect the intended path of movement of the suture needle and the intended depth of penetration of the suture needle and suture.

FIGS. 3-6B illustrate a method of the invention in which the curved suture needle 11 and suture control apparatus 15 apply a suture to an eyeball 30. The diagrams of FIGS. 2-6 are distorted, out of scale, with a portion of the suture control apparatus 15 adjacent the eye tissue 30 greatly enlarged relative to that tissue in order to illustrate how the depth of penetration of the suture material 12 is determined by the radius of curvature of the suture needle 11 and the suture control apparatus 15.

FIG. 3 illustrates the invention with a thread of suture material 12 attached to the suture needle 11, and the suture controller apparatus 15 seated on tissue such as an eyeball 30. As apparent from FIG. 3, the depth of penetration P of the suture needle 11 and suture material 12 can be controlled by the radius of curvature of the curved suture needle 11 and the pair of curved needle guides 16, 17, the distance between the inner openings 16b, 17b of the curved needle guides 16, 17 that form space 18, and the location and shape of the locator surface 20 between the inner openings 16b, 17b of the curved needle guides 16, 17. In the FIGS. 2-6 diagrams, the locator surface 20 is curved, having substantially the same radius of curvature as the outer surface of the eyeball to provide stability in seating the suture controller apparatus 15 on the eyeball, and consistency in the depth of penetration P of the suture into the eyeball 30. For the same distance of the space 18 between the inner openings 16b, 17b of the needle guides 16, 17, decreasing the radius of curvature of the suture needle and the curved needle guides 16, 17 will increase the depth of penetration P of the suture, and increasing the radius of curvature of the suture needle and curved needle guides will decrease the depth of penetration P of the suture. A pre-selected depth of penetration of the sutures can be controlled by the design of the suture control apparatus 15 and is most easily effected by the displacement of the locator surface 20 with respect to from the inner openings 16b, 17b and the path formed by curved needle guides 16, 17.

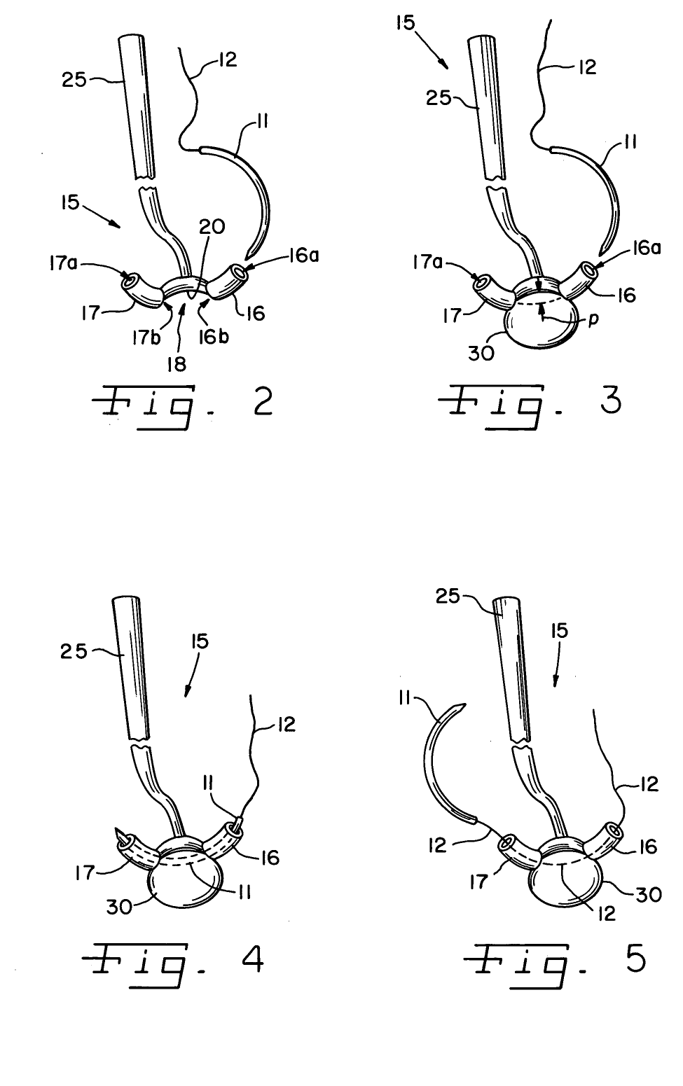

As illustrated by FIGS. 3-6B, the method of the invention is effected by providing a curved suture needle 11 having a radius of curvature, with thread-like suture material 12 attached at the blunt end of the curved suture needle 11; providing, for use with the curved suture needle 11 and attached suture material 12, a suture control apparatus 15 as illustrated in FIGS. 1 and 2; pressing the locator surface 20 of the suture control apparatus 15 against the tissue 30 to receive a suture, with the inner openings 16b, 17b of the pair of curved tubular needle guides 16, 17 located adjacent the portion of the tissue 30 to receive a suture, as illustrated in the FIG. 3 diagram; threading the suture needle 11 through the pair of curved needle guides 16, 17 and the tissue 30 between the inner openings 16b, 17b, as illustrated in the FIG. 4 diagram, so the sharpened end of the suture needle 11 can be used to pull the suture material 12 through the tissue 30 as illustrated in the FIG. 5 diagram. Upon completion of the suture, the suture control apparatus 15 is removed from the suture material 12 through the use of the slots 16c, 17c formed in the curved tubular needle guides 16, 17, as illustrated in the expanded diagrams FIGS. 6A and 6B. If advisable, the surgeon may make multiple loops through the tissue being attached by passing the curved suture needle 11 and with its attached suture material 12 repeatedly through the curved needle guides 16, 17, and upon completion of each of the multiple passes, the suture controller apparatus 15 can be removed by passing the suture material 12 through the slots 16c, 17c.

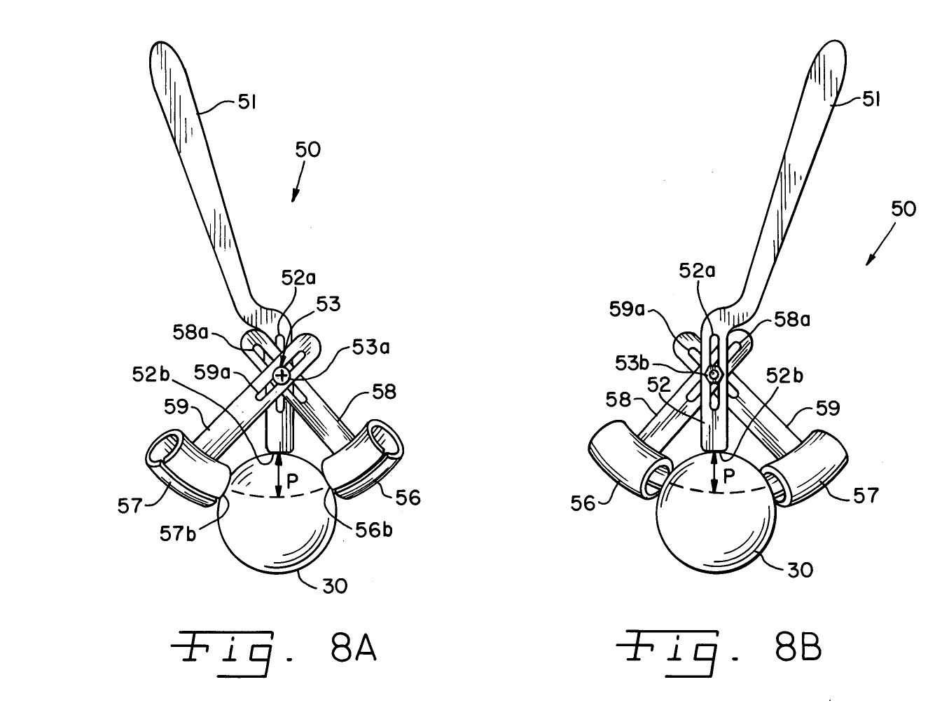

FIGS. 7A-7C illustrate other embodiments of the invention in which the passageways of the curved needle guides 46, 47 of the suture control apparatus 15, and the curved needle 37 have non-circular cross-sections. To avoid a displacement of the sutures from their intended location, which may result because of the slight rotation of a curved suture needle having a circular cross-section within the circular cross-section passageways of the curved tubular needle guides, such as 16, 17 illustrated FIGS. 1-6B, the curved suture needle and the passageways of the curved needle guides may be formed with non-circular cross-sections. For example, the curved suture needle and passageways of the curved needle guides may have cross-sections that are elliptical or polygonal, e.g., four-sided, trapezoidal or square, as illustrated in FIGS. 7A and 7C, triangular, as illustrated in FIG. 7B, or combinations of curved and straight perimetral portions

FIGS. 8A and 8B illustrate a further embodiment of the invention in which the suture control apparatus 50 can be adjusted to provide variation in the controlled depth of penetration of a suture needle and suture. The suture control apparatus 50 includes a pair of needle guides 56, 57 that are carried at the ends of a pair of needle guide arms 58, 59, and handle 51 that includes a tissue engaging end 52 that includes a slot 52a and a locater surface 52b. As illustrated in FIGS. 8A and 8B, a fastener 53 passes through the slots 58a, 59a in the needle guide arms 58, 59 and slot 52a in the tissue engaging end 52 of the handle 51, and fastener 53 provides an axle, whose distance from the locator surface 52b can be adjusted within slot 52a and upon which the needle guide arms 58, 59 may be adjusted in length and pivoted. The fastener 53 may comprise a screw 53a and a nut 53b that may be loosened and tightened and permit 1) adjustment of the lengths of the needle guide arms 58, 59 which can permit adjustment of the radius of curvature of the intended path of a suture and adjustment of the distance between the inner ends 56b, 57b of the needle guides 56, 57 and adjustment of the displacement P of the intended path of a suture needle from the locator surface 52b, and/or 2) the angle between the needle guide arms 58, 59 which can permit an adjustment of the distance between the inner ends 56b, 57b of the needle guides and adjustment thereby of the displacement P of the intended path of a suture needle and suture from the locator surface 52b, and/or 3) adjustment of the distance between the axle 53 and the locator surface 52b which can permit, with or without the adjustments 1 and/or 2, adjustment of the displacement P of the intended path of the suture needle and suture. Slots 58a, 59a and 52a can be any selected length, but it is generally preferable that their lengths be a fraction of an inch, for example, about ¼ inch to about ½ inch in length. Their widths are preferably selected to accept a common screw size.

The foregoing description and drawings of presently preferred embodiments is illustrative of the invention rather than limiting. Those skilled in the art will recognize that the invention can include embodiments other than those illustrated and described above without departing from the scope of the following claims, including all equivalents.

I claim:

1. An apparatus for suturing tissue, comprising
a curved suture needle having a radius of curvature; and
a suture control apparatus comprising a pair of needle guides separated by a space and a locator surface in the space between the needle guides for engagement with tissue to receive a suture, said needle guides providing substantially the same radius of curvature as the radius of curvature of said suture needle, the space between said needle guides and the location of said locator surface being selected to define the depth of the suture through tissue pressed against the locator surface in the space between the needle guides when the curved suture needle is passed through the needle guides and the tissue.

2. The apparatus of claim 1 wherein the curved needle guides have lengths of a small fraction of an inch.

3. The apparatus of claim 1 wherein the needle guides are curved and formed by lengths of tubing.

4. The apparatus of claim 3, wherein the cross-section of the curved suture needle and the cross-sections of the passageways of the curved tubular guides are non-circular.

5. The apparatus of claim 4, wherein the cross-sections of the curved suture needle and of the passageways of the curved needle guides are polygonal.

6. The apparatus of claim 5, wherein the cross-sections of the curved suture needle and of the passageways of the curved needle guides are triangular.

7. The apparatus of claim 3, wherein the cross-sections of the curved suture needle and the passageways of the curved needle guides include a portion formed by a straight line.

8. The apparatus of claim 1, wherein the locator surface is curved.

9. The apparatus of claim 8, wherein the locator surface has substantially the same radius of curvature as a human eyeball.

10. The apparatus of claim 1 wherein the pair of needle guides and locator surface are carried by a handle.

11. The apparatus of claim 10 wherein the pair of needle guides are located at the ends of a pair of needle guide arms pivotally carried by the handle adjacent the locator surface, and wherein a fastener forms an axle on which the pair of needle guide arms are pivotally carried and permits adjustment of the angle between the needle guide arms and the space between the needle guides.

12. The apparatus of claim 11 wherein each of the pair of needle guide arms includes a slot in which the fastener is located and wherein the fastener also permits adjustment in the lengths of the needle guide arms.

13. The apparatus of claim 10 wherein the handle includes a slot adjacent the locator surface, wherein the pair of needle guides are carried by a fastener located within the slot, and wherein the fastener permits adjustment of the location of the needle guides with respect to the location surface

14. The apparatus of claim 13 wherein each of the pair of needle guides is carried at the end of a needle guide arm and the pair of needle guide arms are pivotally carried by the fastener.

15. The apparatus of claim 14 wherein each of the pair of needle guide arms includes a slot at its end distal from the needle guide and the fastener is located within the slot of the needle guide arm permitting adjustment of the lengths of the needle guide arms.

16. The apparatus of claim 13 wherein each of the pair of needle guide arms includes a slot at its end distal from the needle guide and wherein the fastener is located in the slots of the needle guide arms, permitting adjustment in the lengths of the needle guide arms.

17. The apparatus of claim 10 wherein at least one of the needle guides is carried by a needle guide arm with a slot therein and wherein a fastener is carried by the handle and located within the slot of the needle guide arm and permits adjustment of the position of the needle guide.

18. A method of providing sutures having a predetermined depth of penetration in tissue, comprising
providing a curved suture needle having a radius of curvature and an attached suture material;
providing a suture control apparatus for the curved suture needle comprising a pair of curved needle guides separated by a space with a locator surface at a pre-selected site in the space between the pair of curved needle guides, said curved needle guides having the substantially same radius of curvature as the curved suture needle;
pressing the locator surface of the suture controller apparatus against tissue to receive a suture, and locating the pair of curved needle guides adjacent the portion of the tissue to receive the suture;
threading the suture needle and suture material through the pair of curved needle guides and the tissue therebetween to provide a suture having a predetermined depth of penetration of the tissue determined by the radius of curvature of the suture needle, the distance across the space between the pair of curved needle guides and the location of the locator surface.

19. The method of claim 18, wherein the cross-section of the curved suture needle and of the cross-sections of the passageways of the curved needle guides are non-circular.

20. The method of claim 19, wherein the cross-sections of the curved suture needle and of the passageways of the curved needle guides are polygonal.

21. The method of claim 19, wherein the cross-sections of the curved suture needle and of the passageways of the curved tubular needle guides are triangular.

22. The method of claim 18, wherein the locator surface is curved.

23. The method of claim 22, wherein the locator surface has substantially the same radius of curvature as a human eyeball, and is pressed against the outside surface of the eyeball to provide a suture through the tissue of the eyeball having a limited depth of penetration.

24. The method of claim 18 wherein the pair of curved needle guides include slots and the suture central apparatus is manipulated, after application of a suture to tissue, to remove the suture material from the suture control apparatus through the slots.

25. An apparatus for suturing tissue, comprising
a curved suture needle having a radius of curvature; and
a suture control apparatus comprising a pair of curved needle guides that are formed from slotted tubing that is a small fraction of an inch in length and separated by a space, and a locator surface in the space between the curved needle guides for engagement with tissue to receive a suture, said curved tubular needle guides and said curved suture needle having substantially the same radius of curvature, the radius of curvature of said suture needle, the space between said curved tubular needle guides and the location of said locator surface being selected to define the depth of the suture through tissue pressed against the locator surface in the space between the curved tubular needle guides when the curved suture needle is passed through the curved needle guides and the tissue.

26. The apparatus of claim 25, wherein the cross-section of the curved suture needle and the cross-sections of the passageways of the curved tubular needle guides are non-circular.

27. The apparatus of claim 26, wherein the cross-sections of the curved suture needle and of the passageways of the curved needle guides include a portion formed by a straight line.

28. The apparatus of claim 25, wherein the locator surface is curved and has substantially the same radius of curvature as a human eyeball.

29. The apparatus of claim 25, wherein the cross-section of the curved suture needle and of the passageways of the curved needle guides are trapezoidal.
